(19) United States
(12) Patent Application Publication
(10) Pub. No.: US 2015/0140344 A1
(43) Pub. Date: May 21, 2015

(54) COATED MEDICAL DEVICES

(71) Applicant: COVIDIEN LP, Mansfield, MA (US)

(72) Inventors: Xiaodong Ma, Hudson, MA (US); Junwei Li, Irvine, CA (US); Min-Shyan Sheu, Chelmsford, MA (US); Anastasia Righter, Milford, MA (US); Ariana Gilmore, Mansfield, MA (US)

(21) Appl. No.: 14/085,127

(22) Filed: Nov. 20, 2013

(51) Int. Cl. A61L 31/10

(52) U.S. Cl. CPC A61L 31/10
USPC 428/447; 427/2.3

(57) ABSTRACT

Processes for coating medical devices are provided herein. The processes include contacting a substrate with a sulfur-functional silane and an additional silane. The resulting coating includes a silane layer having improved adherence to the medical device, covering a greater area.

### TECHNICAL FIELD

The present disclosure relates to coated and/or surface-treated medical devices. More particularly, the present disclosure relates to methods for coating medical implants, in embodiments implants made of inert materials such as metals, by immobilizing combinations of silanes on surfaces of the implants.

### BACKGROUND

Techniques for repairing damaged or diseased tissue are widespread in medicine. Vascular disease includes aneurysms which can rupture and cause hemorrhage, as well as atherosclerosis, which can cause the occlusion of blood vessels, vascular malformation and tumors. Devices for treating vascular occlusions include implants such as stents, which are placed in the occluded region of the blood vessel to hold it open. Stents of various construction may be utilized, including balloon expandable metal stents, self-expanding braided metal stents, knitted metal stents, coiled stents, rolled stents, and the like. Stent-grafts are also used, which include tubular graft material supported by a metallic stent.

Coatings have been applied to medical devices to impart lubricious, non-thrombogenic, and/or anti-adhesive properties and serve as depots for bioactive agent release. Adherence of these coatings to the substrate used to form the device may prove difficult, with delamination occurring in some cases. In addition, where the substrate to be coated is a metal or similar inert material, conventional processes may require the use of strong bases, acids, or similar oxidizers to generate hydroxyl groups to which the outer coating or any intermediate layer of the coating may bind. However, certain metals, such as platinum, are hard to hydroxylate, which may increase problems with coating formation and/or adherence.

Improved coatings for medical devices, and processes for their application, thus remain desirable.

### SUMMARY

The present disclosure provides medical devices and methods for forming layers on such devices. In embodiments, a medical device of the present disclosure includes a substrate, a silane layer including at least one sulfur-functional silane and at least one additional silane, on at least a portion of the substrate, and at least one additional component bound to the silane layer, the at least one additional component including monomers, polymers, bioactive agents, and combinations thereof.

In embodiments, the substrate used to form the medical device includes an inert material such as glass, ceramics, and metals. Suitable metal substrates include gold, silver, copper, steel, aluminum, cobalt, chromium, platinum, titanium, niobium, tantalum, alloys thereof, and combinations thereof.

Suitable sulfur-functional silanes include bis-[triethoxysilylpropyl]tetrasulfide, 3-mercaptopropyltriethoxysilane, 2,2-dimethoxy-1-thia-2-silacyclo-pentane, 11-mercaptoundecyltrimethoxysilane,s-(octanoyl)mercaptopropyl-triethoxysilane, 2-(2-pyridylethyl)thiopropyltri-methoxysilane, 2-(4-pyridylethyl)thiopropyltri-methoxysilane, 3-thiocyanatopropyltriethoxysilane, 2-(3-trimethoxysilylpropylthio)-thiophene, mercaptomethylmethyldiethoxy-silane, 3-mercaptopropylmethyldimethoxy-silane, bis[3-(triethoxysilyl)propyl]-disulfide, bis-[m-(2-triethoxysilylethyl)tolyl]-polysulfide, bis[3-(triethoxysilyl)propyl]thio-urea, bis-triethoxy silyl propyl polysulfide, and combinations thereof.

In embodiments, the at least one additional silane possesses functional groups such as acrylates, methacrylates, aldehydes, amino, epoxy, esters, anhydride, azide, carboxylate, phosphonate, sulfonate, halogen, hydroxyl, isocyanate, masked isocyanate, phosphine, phosphate, vinyl, olefin, dipodal silanes, UV active components, fluorescent components, chiral components, biomolecular probes, silyl hydrides and combinations thereof.

The at least one sulfur-functional silane is present in amounts from about 0.5% to about 95% by weight of the silane layer, and the at least one additional silane is present in amounts from about 99.5% to about 5% by weight of the silane layer.

In embodiments, the at least one additional component bound to the silane layer may be betaines, phosphorylcholines, [2-(Methacryloyloxy)ethyl]dimethyl-(3-sulfopropyl)ammonium hydroxide, 3-(Methacryloylamino)propyl]dimethyl(3-sulfopropyl)ammonium hydroxide inner salt, and combinations thereof. In other embodiments, the at least one additional component bound to the silane layer includes a phosphorylcholine such as 2-methacryloyloxyethyl phosphorylcholine, 2-acryloyloxyethyl phosphorylcholine, 2-acryloyloxyethyl phosphorylcholine, 2-(meth)acryloyloxyethyl-2′-(trimethylammonio)ethyl phosphate, 3-(meth)acryloyloxypropyl-2′-(trimethylammonio)ethyl phosphate, 4-(meth)acryloyloxybutyl-2′-(trimethylammonio)ethyl phosphate, 5-(meth)acryloyloxypentyl-2′-(trimethylammonio)ethyl phosphate, 6-(meth)acryloyloxyhexyl-2′-(trimethylammonio)ethyl phosphate, 2-(meth)acryloyloxyethyl-2′-(triethylammonio)ethyl phosphate, 2-(meth)acryloyloxyethyl-2′-(tripropylammonio)ethyl phosphate, 2-(meth)acryloyloxyethyl-2′(tributylammonio)ethyl phosphate, 2-(meth)acryloyloxypropyl-2′-(trimethylammonio)ethyl phosphate, 2-(meth)acryloyloxybutyl-2′-(trimethylammonio)ethyl phosphate, 2-(meth)acryloyloxypentyl-2′-(trimethylammonio)ethyl phosphate, 2-(meth)acryloyloxyhexyl-2′-(trimethylammonio)ethyl phosphate, 2-(meth)acryloyloxyethyl-3′-(trimethylammonio)propyl phosphate, 3-(meth)acryloyloxypropyl-3′-(trimethylammonio)propyl phosphate, 4-(meth)acryloyloxybutyl-3′-(trimethylammonio)propyl phosphate, 5-(meth)acryloyloxypentyl-3′-(trimethylammonio)propyl phosphate, 6-(meth)acryloyloxyhexyl-3′-(trimethylammonio)propyl phosphate, 2-(meth)acryloyloxyethyl-4′-(trimethylammonio)butyl phosphate, 3-(meth)acryloyloxypropyl-4′-(trimethylammonio)butyl phosphate, 4-(meth)acryloyloxybutyl-4′-(trimethylammonio)butyl phosphate, 5-(meth)acryloyloxypentyl-4′-(trimethylammonio)butyl phosphate, 6-(meth)acryloyloxyhexyl-4′-(trimethylammonio)butylphosphate, and combinations thereof.

In embodiments, the at least one additional component is chemically bonded to the silane layer. In other embodiments, the at least one additional component may be covalently bonded to the silane layer.

The at least one additional component may include a bioactive agent such as antimicrobials, analgesics, anesthetics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, steroids, lipids, lipopolysaccharides, polysaccharides, enzymes, and combinations thereof.

Medical devices of the present disclosure include stents, filters, stent coatings, grafts, catheters, stent/grafts, clips and other fasteners, staples, sutures, pins, screws, prosthetic devices, drug delivery devices, anastomosis rings, surgical blades, contact lenses, intraocular lenses, surgical meshes, knotless wound closures, sealants, adhesives, intraocular lenses, anti-adhesion devices, anchors, tunnels, bone fillers, synthetic tendons, synthetic ligaments, tissue scaffolds, stapling devices, buttresses, lapbands, orthopedic hardware, pacers, pacemakers, and implants.

Methods of the present disclosure include methods for forming a silane layer on a surface of a medical device. The methods include, in embodiments, contacting the surface of the medical device with at least one sulfur-functional silane and at least one additional silane to form the silane layer, and contacting the silane layer with at least one additional component such as monomers, polymers, bioactive agents, and combinations thereof.

The at least one sulfur-functional silane utilized in the disclosed methods may be bis-[triethoxysilylpropyl]tetrasulfide, 3-mercaptopropyltriethoxysilane, 2,2-dimethoxy-1-thia-2-silacyclo-pentane, 11-mercaptoundecyltrimethoxysilane, s-(octanoyl)mercaptopropyl-triethoxysilane, 2-(2-pyridylethyl)thiopropyltri-methoxysilane, 2-(4-pyridylethyl)thiopropyltri-methoxysilane, 3-thiocyanatopropyltriethoxysilane, 2-(3-trimethoxysilylpropylthio)-thiophene, mercaptomethylmethyldiethoxy-silane, 3-mercaptopropylmethyldimethoxy-silane, bis[3-(triethoxysilyl)propyl]-disulfide, bis-[m-(2-triethoxysilylethyl)tolyl]-polysulfide, bis[3-(triethoxysilyl)propyl]thio-urea, bis-triethoxy silyl propyl polysulfide, and combinations thereof.

The at least one additional silane utilized in the disclosed methods may possess functional groups such as acrylates, methacrylates, aldehydes, amino, epoxy, esters, anhydride, azide, carboxylate, phosphonate, sulfonate, halogen, hydroxyl, isocyanate, masked isocyanate, phosphine, phosphate, vinyl, olefin, dipodal silanes, UV active components, fluorescent components, chiral components, biomolecular probes, silyl hydrides, and combinations thereof.

In embodiments, the method of the present disclosure also includes hydroxylating the surface of the medical device prior to contacting the surface with the at least one sulfur-functional silane and the at least one additional silane. The surface of the medical device may be hydroxylated by subjecting the surface to a treatment with sodium hydroxide, nitric acid, sulfuric acid, hydrochloric acid, ammonium hydroxide, hydrogen peroxide, tert-butyllyn hydroperoxide, potassium dichromate, perchloric acid, oxygen plasma, water plasma, corona discharge, ozone, UV, and combinations thereof.

In embodiments, the methods of the present disclosure result in the at least one sulfur-functional silane being present in amounts from about 0.5% to about 95% by weight of the silane layer, and the at least one additional silane present in amounts from about 99.5% to about 5% by weight of the silane layer.

In embodiments, the at least one sulfur-functional silane and the at least one additional silane may be sequentially applied. In other embodiments, the at least one sulfur-functional silane and the at least one additional silane are applied in a single mixture.

In embodiments, the at least one additional component bound to the silane layer pursuant to the methods of the present disclosure may be betaines, phosphorylcholines, [2-(Methacryloyloxy)ethyl]dimethyl-(3-sulfopropyl)ammonium hydroxide, 3-(Methacryloylamino)propyl]dimethyl(3-sulfopropyl)ammonium hydroxide inner salt, and combinations thereof. In other embodiments, the at least one additional component bound to the silane layer includes a phosphorylcholine such as 2-methacryloyloxyethyl phosphorylcholine, 2-acryloyloxyethyl phosphorylcholine, 2-acryloyloxyethyl phosphorylcholine, 2-(meth)acryloyloxyethyl-2′-(trimethylammonio)ethyl phosphate, 3-(meth)acryloyloxypropyl-2′-(trimethylammonio)ethyl phosphate, 4-(meth)acryloyloxybutyl-2′-(trimethylammonio)ethyl phosphate, 5-(meth)acryloyloxypentyl-2′-(trimethylammonio)ethyl phosphate, 6-(meth)acryloyloxyhexyl-2′-(trimethylammonio)ethyl phosphate, 2-(meth)acryloyloxyethyl-2′-(triethylammonio)ethyl phosphate, 2-(meth)acryloyloxyethyl-2′-(tripropylammonio)ethyl phosphate, 2-(meth)acryloyloxyethyl-2′-(tributylammonio)ethyl phosphate, 2-(meth)acryloyloxypropyl-2′-(trimethylammonio)ethyl phosphate, 2-(meth)acryloyloxybutyl-T-(trimethylammonio)ethyl phosphate, 2-(meth)acryloyloxypentyl-2′-(trimethylammonio)ethyl phosphate, 2-(meth)acryloyloxyhexyl-2′-(trimethylammonio)ethyl phosphate, 2-(meth)acryloyloxyethyl-3′-(trimethylammmonio)propyl phosphate, 3-(meth)acryloyloxypropyl-3′-(trimethylammonio)propyl phosphate, 4-(meth)acryloyloxybutyl-3′-(trimethylammonio)propyl phosphate, 5-(meth)acryloyloxypentyl-3′-(trimethylammonio)propyl phosphate, 6-(meth)acryloyloxyhexyl-3′-(trimethylammonio)propyl phosphate, 2-(meth)acryloyloxyethyl-4′-(trimethylammonio)butyl phosphate, 3-(meth)acryloyloxypropyl-4′-(trimethylammonio)butyl phosphate, 4-(meth)acryloyloxybutyl-4′-(trimethylammonio)butyl phosphate, 5-(meth)acryloyloxypentyl-4′-(trimethylammonio)butyl phosphate, 6-(meth)acryloyloxyhexyl-4′-(trimethylammonio)butylphosphate, and combinations thereof.

In embodiments, the at least one additional component is chemically bonded to the silane layer. In other embodiments, the at least one additional component is covalently bonded to the silane layer.

In embodiments, the at least one additional component utilized in the methods of the present disclosure includes a bioactive agent such as antimicrobials, analgesics, anesthetics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, steroids, lipids, lipopolysaccharides, polysaccharides, enzymes, and combinations thereof.

Medical devices treated by the methods of the present disclosure includes stents, filters, stent coatings, grafts, catheters, stent/grafts, clips and other fasteners, staples, sutures, pins, screws, prosthetic devices, drug delivery devices, anastomosis rings, surgical blades, contact lenses, intraocular lenses, surgical meshes, knotless wound closures, sealants, adhesives, intraocular lenses, anti-adhesion devices, anchors, tunnels, bone fillers, synthetic tendons, synthetic ligaments, tissue scaffolds, stapling devices, buttresses, lapbands, orthopedic hardware, pacers, pacemakers, and implants.

### BRIEF DESCRIPTION OF THE DRAWINGS

The accompanying drawings, which are incorporated in and constitute a part of this specification, illustrate embodiments of the disclosure and, together with the general description of the disclosure given above, and the detailed description of the embodiment(s) given below, serve to explain the principles of the disclosure, wherein:

FIG. 1 is a schematic diagram showing a general example of the chemical structure of sulfidosilanes.

FIGS. 2, 3, 4 and 5 are graphs showing the results of X-ray photoelectron spectroscopy performed on metallic sheets coated by methods of the present disclosure.

FIG. 6 is a graph depicting the lag time before thrombin formed on stents possessing a coating of the present disclosure, compared to bare stents not possessing any coating, glass beads (as a positive control), and polystyrene wells (as a negative control);

FIG. 7 is a graph depicting peak amounts of thrombin formed on stents possessing a coating of the present disclosure, compared to bare stents not possessing any coating, glass beads, and polystyrene wells; and

FIG. 8 is a graph depicting time to peak thrombin formation on stents possessing a coating of the present disclosure, compared to bare stents not possessing any coating, glass beads, and polystyrene wells.

### DETAILED DESCRIPTION

The present disclosure provides devices having coatings and/or layers thereon, as well as embodiments for applying coatings/layers to medical devices. Substrates used to form medical devices in accordance with the present disclosure may be formed of any suitable substance, including inert materials such as metals, glass, ceramics, combinations thereof, and the like.

In embodiments, substrates of the present disclosure may be formed of inert materials such as glass, ceramics, and/or metals. Suitable metals include, but are not limited to, for example, gold, silver, copper, steel, aluminum, cobalt, chromium, platinum, titanium, niobium, and tantalum, alloys thereof, combinations thereof, and the like. Suitable alloys include cobalt-nickel, cobalt-chromium, and platinum-tungsten. In embodiments, a suitable cobalt based alloy is 35N LT™ available from Fort Wayne Metals of Fort Wayne, Ind. USA.

The medical devices of the present disclosure include a coating formed, at least in part, by a silane layer. The present disclosure utilizes silanes possessing sulfur groups to form either an outer layer on the medical device, or an intermediate layer which binds to the metal substrate. In embodiments, additional layers, including polymer layers, layers of bioactive agents, combinations thereof, and the like, may then be applied to and bound to the silane layer.

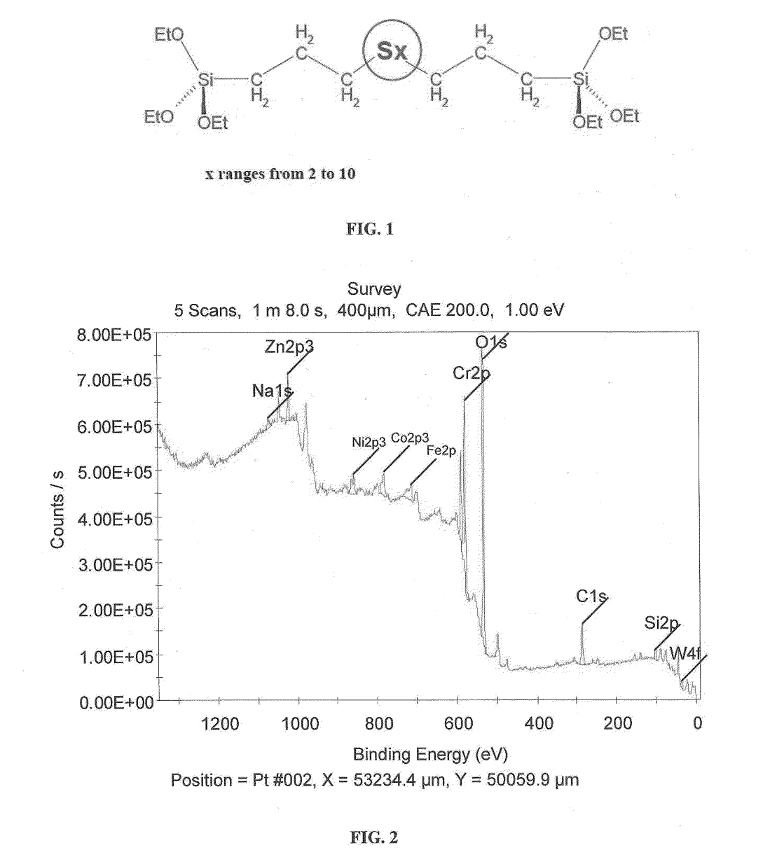

In embodiments, the silane layer includes sulfur-functional silanes. Suitable sulfur-functional silanes include, but are not limited to, for example, multifunctional sulfur silanes. In embodiments, suitable sulfur-functional silanes include bis-[triethoxysilylpropyl]tetrasulfide (TS), 3-mercaptopropyltriethoxysilane, 2,2-dimethoxy-1-thia-2-silacyclo-pentane, 11-mercaptoundecyltrimethoxysilane, s-(octanoyl)mercaptopropyl-triethoxysilane, 2-(2-pyridylethyl)thiopropyltri-methoxysilane, 2-(4-pyridylethyl)thiopropyltri-methoxysilane, 3-thiocyanatopropyltriethoxysilane, 2-(3-trimethoxysilylpropylthio)-thiophene, mercaptomethylmethyldiethoxy-silane, 3-mercaptopropylmethyldimethoxy-silane, bis[3-(triethoxysilyl)propyl]-disulfide, bis-[m-(2-triethoxysilylethyl)tolyl]-polysulfide, bis[3-(triethoxysilyl)propyl]thio-urea, bis-triethoxy silyl propyl polysulfide, combinations thereof, and the like. In embodiments, suitable sulfur-functional silanes include sulfidosilanes. FIG. 1 is a schematic diagram showing a general example of the chemical structure of sulfidosilanes.

In embodiments, the silane layer may include sulfur-functional silanes as described above combined with additional silanes. Additional silanes which may be utilized in forming the silane layer may have at least one functional group including, but not limited to, acrylate, methacrylate, aldehyde, amino, epoxy, ester, anhydride, azide, carboxylate, phosphonate, sulfonate, halogen, hydroxyl, isocyanate, masked isocyanate, phosphine, phosphate, vinyl, olefin, dipodal silanes, UV active components, fluorescent components, chiral components, biomolecular probes, silyl hydrides, combinations thereof, and the like. In embodiments, additional silanes which may be used in forming the silane layer include, but are not limited to, 3-glycidyloxypropyl trimethoxysilane (OPTS), 2-bromo-2-methyl-N-3-[(trimethoxysilyl)propyl]-propanamide (BrTMOS), combinations thereof, and the like.

Where multiple silanes are used to form a silane layer of the present disclosure, the silanes may be applied as a mixture, or the silanes may be applied sequentially.

Where combinations of silanes are used to form silane layers, the sulfur-functional silanes may be present in amounts from about 0.5% to about 95% by weight of the silane layer, in embodiments from about 5% to about 50% by weight of the silane layer, with the additional silane present in amounts from about 99.5% to about 5% by weight of the silane layer, in embodiments from about 95% to about 50% by weight of the silane layer.

The ratio of sulfur-functional silane to additional silane may be from about 1:200 to about 19:1, in embodiments about 1:3 sulfur-functional silane to additional silane.

In embodiments, the silanes used to form the silane layer may be in solution, which is then applied to the substrate. Suitable solvents for forming the solution include but are not limited to, for example, ethanol, water, deionized water, methanol, isopropyl alchohol, n-butanol, dimethylformamide (DMF), dimethyl sulfoxide (DMSO), ethyl acetate, propylene glycol monomethyl ether acetate (PM acetate), toluene, chloroform, dichloromethane, combinations thereof, and the like. The solvents may be present in amounts from about 0.1% to about 99.9% by weight of the solution, in embodiments from about 0.5% to about 95% by weight of the solution. In some embodiments, the solution may include ethanol and water at a ratio from about 95%/5%.

In embodiments, a suitable solution for applying a silane layer may include 0.667% TS and 2% GPTS in 95%/5% ethanol/water with a ratio of TS:GPTS of 1:3.

To accelerate the process of silanization, an acid or base may be added into the aforementioned silane solutions to adjust the solution's pH. Suitable acids or bases include, but are not limited to, for example, acetic acid, nitric acid, sulfuric acid, hydrochloric acid, perchloric acid, sodium hydroxide, potassium hydroxide, barium hydroxide, ammonium hydroxide, combinations thereof, and the like.

To apply the silane layer to the substrate, it may be desirable to first clean the substrate surface. For example, the substrate surface may first be subjected to sonication and cleaned with a suitable solvent such as acetone, isopropyl alcohol, ethanol, methanol, isopropyl alchohol, n-butanol, alcohols, dimethylformamide (DMF), dimethyl sulfoxide (DMSO), ethyl acetate, PM acetate, toluene, chloroform, dichloromethane, combinations thereof, and the like. Sonication may occur for a period of time from about 1 minute to about 60 minutes, in embodiments from about 5 minutes to about 15 minutes. The solvents used in the sonication/cleaning may be applied as mixtures, or individual solvents may be applied sequentially, one or more times. The sonication may occur at temperatures from about 18° C. to about 55° C., in embodiments from about 40° C. to about 50° C., in embodiments about 45° C.

After cleaning, the substrates may be subjected to a treatment to enhance the formation of hydroxyl groups (sometimes referred to, herein, as hydroxylation). Materials used for hydroxylation include, but are not limited to, hydroxyides, such as sodium hydroxide, nitric acid, sulfuric acid, hydrochloric acid, ammonium hydroxide, hydrogen peroxide, tert-butyl hydroperoxide, potassium dichromate, perchloric acid, as well as oxygen plasma, water plasma, corona discharge, ozone, UV, combinations thereof, and the like. The hydroxide may be at a concentration from about 10% to about 98%, in embodiments from about 15% to about 25%, in embodiments about 20%. Hydroxylation may occur over a period of time from about 5 minutes to about 24 hours, at a temperature from about 20° C. to about 80° C., in embodiments from about 1 hour to about 2 hours, in embodiments about 1.5 hours, at room temperature. Hydroxylation may also occur with shaking or stirring from about 30 to about 300 revolutions per minute (rpm), in embodiments from about 120 to about 140 rpm, in embodiments about 130 rpm.

After hydroxylation, the substrate may be rinsed with a suitable material, such as deionized water.

The hydroxylated substrate may then be treated with the silanes described above. The sulfur-functional silane and additional silane may be applied as a mixture as described above or, in other embodiments, may be applied sequentially. For example, in embodiments, the substrate may be immersed in a solution including both the sulfur-functional silane and additional silane for a period of time from about 5 minutes to about 24 hours, in embodiments from about 1 hour to about 3 hours, in embodiments for about 2 hours, at room temperature.

In other embodiments, the substrate may be immersed in the sulfur-functional silane for a period of time from about 5 minutes to about 24 hours, in embodiments from about 0.5 hours to about 1.5 hours, in embodiments for about 1 hour, followed by immersion of the substrate in the additional silane for a period of time from about 5 minutes to about 24 hours, in embodiments from about 0.5 hours to about 1.5 hours, in embodiments for about 1 hour. This sequential immersion may also occur at room temperature.

After immersion in the silane materials, the substrate may then be rinsed with, or immersed into, a suitable material, such as ethanol, acetone, isopropyl alcohol, methanol, isopropyl alchohol, n-butanol, alcohols, dimethylformamide (DMF), dimethyl sulfoxide (DMSO), ethyl acetate, PM acetate, toluene, chloroform, dichloromethane, combinations thereof, and the like, from one time to about 5 times, in embodiments about 3 times. If immersed, the immersion time may be from about 10 seconds to about 1 minute. The substrate with the silanes thereon may then be heated at a temperature from about 30° C. to about 150° C., in embodiments from about 60° C. to about 120° C., in embodiments about 80° C. Heating may occur for a time from about 2 minutes to about 120 minutes, in embodiments from about 5 minutes to about 30 minutes.

Once the silane layer has been formed, the medical device may be treated with additional components to form an outer layer on the silane layer. For example, bioactive agents may bind to free functional groups of the silane layer. Similarly, polymeric and/or monomeric materials may bind free functional groups of the silane layer, with or without bioactive agents.

Suitable polymeric and/or monomeric materials which may be utilized to form an outer coating on the medical device of the present disclosure, binding to the silane layer described above, include any material suitable for use in the medical device to be coated. Such materials may provide desirable properties to the medical device, including cell and protein adhesion, lubricity, drug delivery, cell delivery, protein delivery, RNA delivery, gene delivery, prevention of restenosis, reduction of thrombogenicity, anti-microbial properties, non-fouling properties, promoting endothelialization, combinations thereof, and the like. Such materials may include any group capable of reacting with the silane layer including, but not limited to, carboxylate, isocyanate, acrylate, methacrylate, aldehyde, epoxy, ester, anhydride, azide, halogen, hydroxyl amine, epoxy, thiol, vinyl, aldehyde, combinations thereof, and the like.

In embodiments, suitable polymeric and/or monomeric materials which may bind to the silane layer and be used to form an outer coating on the medical device of the present disclosure include, for example, betaines, phosphorylcholines, poly(ethylene glycol), polyurethanes, combinations thereof, and the like. Suitable phosphorylcholines include but are not limited to 2-methacryloyloxyethyl phosphorylcholine (MPC), 2-acryloyloxyethyl phosphorylcholine, and the like, and combinations thereof. Other phosphorylcholines may be utilized, including phosphorylcholines based upon monomers including, but not limited to, 2-(meth)acryloyloxyethyl-2′-(trimethylammonio)ethyl phosphate, 3-(meth)acryloyloxypropyl-2′-(trimethylammonio)ethyl phosphate, 4-(meth)acryloyloxybutyl-2′-(trimethylammonio)ethyl phosphate, 5-(meth)acryloyloxypentyl-2′-(trimethylammonio)ethyl phosphate, 6-(meth)acryloyloxyhexyl-T-(trimethylammonio)ethyl phosphate, 2-(meth)acryloyloxyethyl-2′-(triethylammonio)ethyl phosphate, 2-(meth)acryloyloxyethyl-2′-(tripropylammonio)ethyl phosphate, 2-(meth)acryloyloxyethyl-2′-(tributylammonio)ethyl phosphate, 2-(meth)acryloyloxypropyl-2′-(trimethylammonio)ethyl phosphate, 2-(meth)acryloyloxybutyl-2′-(trimethylammonio)ethyl phosphate, 2-(meth)acryloyloxypentyl-2′-(trimethylammonio)ethyl phosphate, 2-(meth)acryloyloxyhexyl-2′-(trimethylammonio)ethyl phosphate, 2-(meth)acryloyloxyethyl-3′-(trimethylammonio)propyl phosphate, 3-(meth)acryloyloxypropyl-3′-(trimethylammonio)propyl phosphate, 4-(meth)acryloyloxybutyl-3′-(trimethylammonio)propyl phosphate, 5-(meth)acryloyloxypentyl-3′-(trimethylammonio)propyl phosphate, 6-(meth)acryloyloxyhexyl-3′-(trimethylammonio)propyl phosphate, 2-(meth)acryloyloxyethyl-4′-(trimethylammonio)butyl phosphate, 3-(meth)acryloyloxypropyl-4′-(trimethylammonio)butyl phosphate, 4-(meth)acryloyloxybutyl-4′-(trimethylammonio)butyl phosphate, 5-(meth)acryloyloxypentyl-4′-(trimethylammonio)butyl phosphate, 6-(meth)acryloyloxyhexyl-4′-(trimethylammonio)butylphosphate, and combinations thereof. As used herein, “(meth)acryl” includes both methacryl and/or acryl groups.

Suitable betaines include, but are not limited to, sulfobetaines, carboxybetaines, phosphorobetaines, and the like. Other suitable materials for use in the outer coating or layer include [2-(methacryloyloxy)ethyl]dimethyl-(3-sulfopropyl)ammonium hydroxide, 3-(methacryloylamino)propyl]dimethyl(3-sulfopropyl)ammonium hydroxide inner salt, combinations thereof, and the like.

Additional materials may bind to the silane layer by chemical bonding, covalent binding, combinations thereof, and the like, depending on the materials selected to bind to the silane layer.

As noted above, bioactive agents may be added to a medical device of the present disclosure, either as part of the device, and/or as part of the coating applied in accordance with the present disclosure. A “bioactive agent,” as used herein, includes any substance or mixture of substances that provides a therapeutic or prophylactic effect; a compound that affects or participates in tissue growth, cell growth and/or cell differentiation; a compound that may be able to invoke or prevent a biological action such as an immune response; or a compound that could play any other role in one or more biological processes. A variety of bioactive agents may be incorporated into the medical device. Moreover, any agent which may enhance tissue repair, limit the risk of restenosis, and modulate the mechanical or physical properties of the medical device, such as a stent, may be added during the preparation of the medical device. In embodiments, the bioactive agent may be added to the polymer used to form the outer coating. In other embodiments, the bioactive agent itself forms the outer coating of the medical device.

Examples of classes of bioactive agents which may be utilized in accordance with the present disclosure include antimicrobials, analgesics, anesthetics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, steroids, lipids, lipopolysaccharides, polysaccharides, and enzymes. It is also intended that combinations of bioactive agents may be used.

Other bioactive agents which may be in the present disclosure include antirestenotic agents, including paclitaxel, paclitaxel derivatives, taxane QP-2, actinomycin D, vincristine, methotrexate, angiopeptin, mitomycin, BCP 678, c-myc antisense sirolimus, sirolimus derivatives, tacrolimus, everolimus, ABT-578, biolimus A9, tranilast, dexamethasone, methylprednisolone, interferon, leflunomide, cyclosporin, halofuginone, c-proteinase inhibitors, metalloproteinase inhibitors, batimastat, propyl hydroxylase inhibitors, VEGF, 17-β-estradiol, BCP 671, HMG CoA reductase inhibitors, combinations thereof, and the like.

Yet other bioactive agents include sympathomimetic agents; vitamins; anticholinergic agents (e.g., oxybutynin); cardiovascular agents such as coronary vasodilators and nitroglycerin; alkaloids; analgesics; non-narcotics such as salicylates, aspirin, acetaminophen, d-propoxyphene and the like; anti-cancer agents; anti-inflammatory agents such as hormonal agents, hydrocortisone, prednisolone, prednisone, non-hormonal agents, allopurinol, indomethacin, phenylbutazone and the like; prostaglandins and cytotoxic drugs; antibacterials; antibiotics; anti-fungals; anti-virals; anticoagulants; and immunological agents.

Other examples of suitable bioactive agents which may be included in the present disclosure include: heparin, hyaluronic acid, collagen, chitin, chitosan, alginate, gelatin, viruses and cells; peptides, polypeptides and proteins, as well as analogs, muteins, and active fragments thereof; immunoglobulins; antibodies; cytokines (e.g., lymphokines, monokines, chemokines); blood clotting factors; hemopoietic factors; interleukins (IL-2, IL-3, IL-4, IL-6); interferons (β-IFN, (α-IFN and γ-IFN)); erythropoietin; nucleases; tumor necrosis factor; colony stimulating factors (e.g., GCSF, GM-CSF, MCSF); insulin; anti-tumor agents and tumor suppressors; blood proteins; gonadotropins (e.g., FSH, LH, CG, etc.); hormones and hormone analogs (e.g., growth hormone); vaccines (e.g., tumoral, bacterial and viral antigens); somatostatin; antigens; blood coagulation factors; growth factors (e.g., nerve growth factor, insulin-like growth factor); protein inhibitors; protein antagonists; protein agonists; nucleic acids such as antisense molecules, DNA, and RNA; oligonucleotides; and ribozymes.

The outer coating(s) (e.g., outer layer(s)) may be applied to the silane layer using any method within the purview of one skilled in the art. For example, the materials used to form the outer coating or layer may be placed in a solution, which may then be applied to the silane layer by dipping, spraying, brushing, combinations thereof, and the like.

In some cases, coatings like phosphorylcholines and/or betaines may be grafted to the medical device by surface-initiated atom transfer radical polymerization (ATRP) onto the silane layer of the present disclosure. These methods are described, for example, in Zhang, et al., “Superlow Fouling Sulfobetaine and Carboxybetaine Polymers on Glass Slides,” Langmuir, (22):10072-10077 (2006), the entire disclosure of which is incorporated by reference herein. Briefly, the substrate surface is modified by a silane compound which contains alkyl halides such as alkyl bromides. The alkyl halides are used to initiate polymerization of acrylate monomers that contain phosphorylcholine and/or betaine. As a result, phosphorylcholine and/or betaine polymers are chemically attached to the substrate, which impart the desired surface properties, such as non-thrombogenicity, lubricity, and biocompatibility. In embodiments of the present disclosure, 2-bromo-2-methyl-N-3-[(trimethoxysilyl)propyl]-propanamide (BrTMOS) may be used to modify the stent surface. The attached alkyl bromides were then used to initiate the polymerzition of phosphorylcholine to produce a phosphorylcholine containing polymer on the substrate which significantly improved the thrombogenic performance of the stent. Further, in accordance with the present disclosure, a sulfur containing silane, bis-[triethoxysilylpropyl]tetrasulfide (TS), was used in combination with BrTMOS. The addition of TS greatly improved the coverage of the BrTMOS and thus the efficiency of surface initiated polymerization.

Suitable medical devices which may be coated in accordance with the present disclosure include, but are not limited to, stents, including braided and non-braided stents such as laser-cut stents, filters, stent coatings, grafts, catheters, stent/grafts, clips and other fasteners, staples, sutures, pins, screws, prosthetic devices, drug delivery devices, anastomosis rings, surgical blades, contact lenses, intraocular lenses, surgical meshes, knotless wound closures, sealants, adhesives, intraocular lenses, anti-adhesion devices, anchors, tunnels, bone fillers, synthetic tendons, synthetic ligaments, tissue scaffolds, stapling devices, buttresses, lapbands, orthopedic hardware, pacers, pacemakers, and other implants and implantable devices.

As noted above, in embodiments, the medical device to be coated or surface-treated in accordance with the present disclosure is a stent. Any stent may be coated or surface-treated in accordance with the methods herein. The stent may be a braided stent or other form of stent such as a laser-cut stent, roll-up stent balloon expandable stent, self-expanding stent, knitted stent, and the like. The stent can optionally be configured to act as a “flow diverter” device for treatment of aneurysms, such as those found in blood vessels including arteries in the brain or within the cranium, or in other locations in the body such as peripheral arteries. The stent can, in embodiments, include those sold as PIPELINE™ Embolization Devices by Covidien, Mansfield, Mass., or other braided stents comprising a number of metallic filaments braided into a tube. One useful combination in constructing such a braided stent includes a first plurality of cobalt-chromium filaments braided with a second plurality of platinum, platinum alloy, or other radiopaque filaments.

Some embodiments of stents disclosed herein can include a self-expanding tube formed from a plurality of filaments that are braided together and define a plurality of pores between the filaments. The stent can include a flow diverting portion having a plurality of pores that have a flow diverting pore size; instead of or in addition to this property, the flow diverting portion can have a flow diverting porosity. The flow diverting portion can include a portion of the stent, or the entire stent. The flow diverting pore size can be an average pore size within a relevant portion of the stent, e.g., within the flow diverting portion or a portion thereof, or a “computed” pore size, one that is computed from measured or nominal basic stent parameters such as braid angle, number of filaments, filament size, filament diameter, stent diameter, longitudinal picks per inch, radial picks per inch, etc. Such a computed pore size can be considered to be one type of average pore size. The flow diverting pore size can be within a size range that that interferes with or inhibits blood flow through the sidewall of the stent, for example, between the parent vessel and an aneurysm sufficient to induce or lead to thrombosis of the aneurysm. The coating or surface treatment can prevail partially or entirely along the flow diverting portion, or along another portion of the stent.

In some embodiments, the pores of the flow diverting portion can have an average pore size of less than 500 microns (inscribed diameter), or from about 20 to about 300 microns (inscribed diameter). Further, the average pore size can be from about 25 to about 250 microns (inscribed diameter). Furthermore, the average pore size can be from about 50 to about 200 microns (inscribed diameter).

In embodiments, most of the accessible surfaces of the stent or other substrate may be covered with the silane layer and optional outer coating. In yet other embodiments, the entire substrate is covered. The coating may cover from about 1% to about 100% of the area of the substrate, in embodiments a stent, in embodiments from about 20% to about 80% of the area of the substrate, and in embodiments from about 40% to about 70% of the area of the substrate.

The processes and coatings of the present disclosure have several advantages. The methods of the present disclosure, using the sulfur-functional silane, enhance adherence of the silane layer to the otherwise inert substrate surface, thereby increasing coverage of silane and desired functional groups on the substrate. Medical devices coated in accordance with the present disclosure thus have both a greater area of coverage, as well as an ability to retain the coating and avoid delamination thereof. Moreover, the methods herein may avoid the hydroxylation step and thus eliminate the need for using strong bases, acids, or other oxidizing agents, and are thus cost-effective and environmentally friendly.

The following Examples are being submitted to illustrate embodiments of the present disclosure. These Examples are intended to be illustrative only and are not intended to limit the scope of the present disclosure. Also, parts and percentages are by weight unless otherwise indicated. As used herein, “room temperature” refers to a temperature of from about 20° C. to about 30° C.

### EXAMPLES

### Example 1

Cobalt chromium alloy (CoCr) and Platinum (Pt) sheets were sonicated/cleaned in acetone (1 time) and isopropyl alcohol (2 times) sequentially for 5 minutes each at 45° C. The sheets were then hydroxylated in a solution of 20% NaOH for 1.5 hours at room temperature with 130 rpm shaking, followed by rinsing in deionized water. The hydroxylated sheets were immersed in a silane solution containing 767 μl (3-Glycidyloxypropyl)trimethoxysilane (GPTS), 250 μl Bis[3-(triethoxysilyl)propyl]tetrasulfide (TS), 28.5 grams absolute ethanol, and 1.5 grams deionized water, and shaken at 130 rpm for 2 hours at room temperature. After that, the sheets were rinsed with absolute ethanol 3 times and cured at 80° C. for 15 minutes. As a control experiment, the Pt and Co—Cr sheets were treated with the same conditions above except TS was not used.

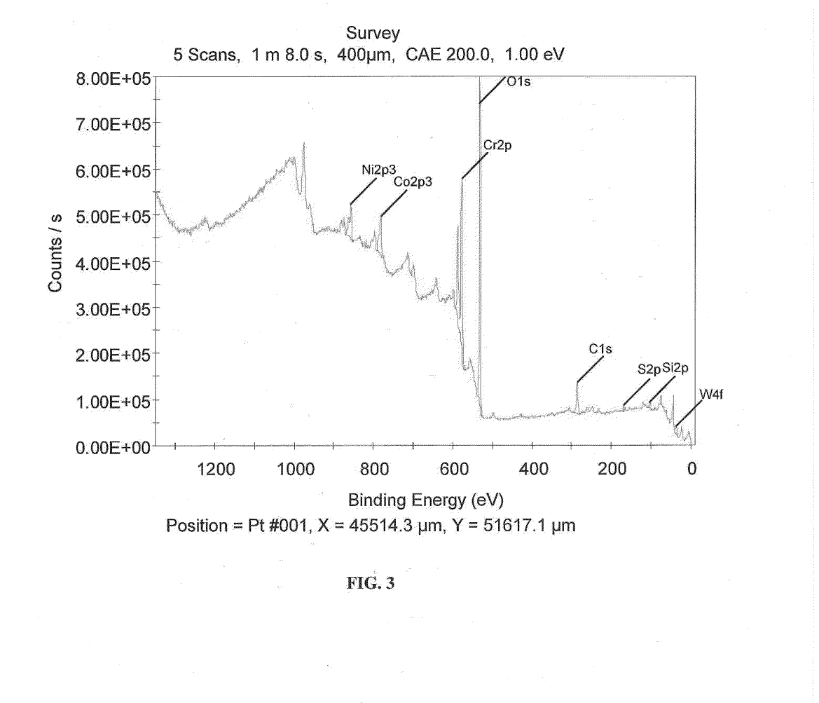

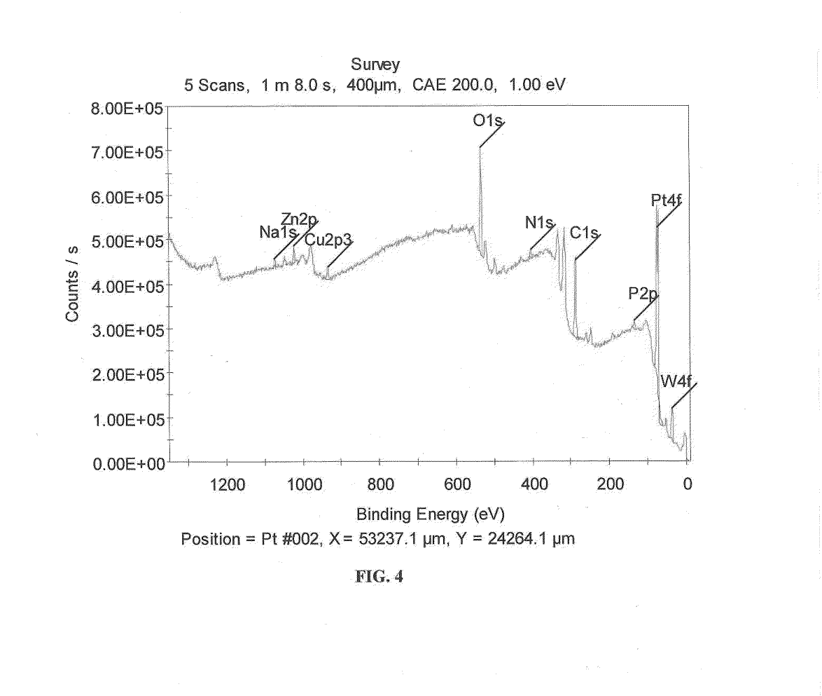

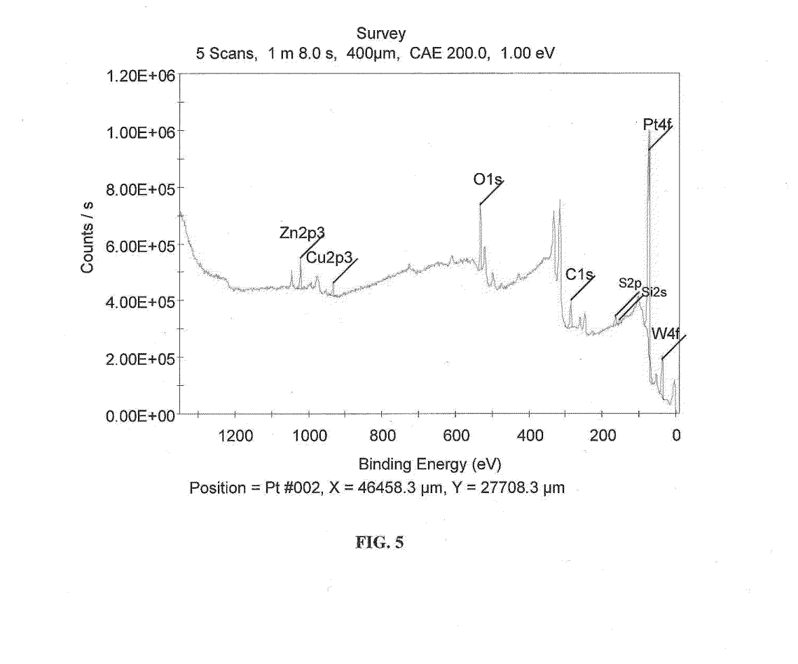

X-ray photoelectron spectroscopy (XPS) was performed on the sheets to measure surface chemical composition. The results pertaining to silicon (Si) element concentration showed that silanization on the Pt sheet surface did not occur when GPTS alone was used in the treatment, while significant amount of silane was attached when TS was used in combination with GPTS. The silanization on the Co—Cr sheet was successful for both OPTS and GPTS-plus-TS treatment, as indicated by the presence of silicon on the surface. The presence of sulfur (5) indicates presence of the TS saline. See FIG. 2 (XPS spectrum of Co—Cr treated with GPTS alone), FIG. 3 (XPS spectrum of Co—Cr treated with GPTS plus TS), FIG. 4 (XPS spectrum of Pt treated with GPTS alone) and FIG. 5 (XPS spectrum of Pt treated with OPTS plus TS). In addition, Tables 1 and 2 below show the corresponding numeric percentages of S and Si in the elemental composition of the sheet surfaces in each of the four XPS measurements referenced above.

TABLE 1

| Elemental Composition of | | |
|---|---|---|
| Co—Cr Sheet Surface, | Treated GPTS | |
| Measured by XPS | Plus TS (%) | Treated GPTS (%) |
| S | 2 | Undetectable |
| Si | 4.22 | 4.64 |

TABLE 2

| Elemental Composition of | | |
|---|---|---|
| Pt Sheet Surface, | Treated GPTS | |
| Measured by XPS | Plus TS (%) | Treated GPTS (%) |
| S | 7.14 | Undetectable |
| Si | 5.6 | Undetectable |

The Pt sheets and CoCr sheets were also subjected to water contact angle tests. Briefly, when a liquid drops on a surface of an object, an angle is formed between the surface and the tangent line of the drop, called contact angle θ. Contact angle is a sensitive method to measure the surface energy and the change of surface chemical composition. If the contact angle is low, the surface has high surface energy and easily gets wet. For example, a contact angle of 0 degrees indicates that water spreads over and becomes a film on the surface so that the surface becomes completely wet, and the surface is less hydrophobic. On the contrary, if the contact angle is high, the surface does not easily get wet and/or may be more hydrophobic. For example, where a surface of a substrate has a contact angle of 180 degrees, the liquid forms spherical droplets and cannot wet the surface at all.

Water droplets were placed on bare CoCr sheets, bare Pt sheets, CoCr sheets treated with NaOH, Pt sheets treated with NaOH, CoCr sheets treated with GPTS/TS as described in this Example, and Pt sheets treated with GPTS/TS as described in this Example. The contact angles are set forth below in Table 3.

TABLE 3

| Sheet | Contact Angle |
|---|---|
| Bare CoCr | 85 ± 3° |
| NaOH treated CoCr | 7 ± 1° |
| GPTS/TS treated CoCr | 49 ± 14° |
| BarePt | 80 ± 6° |
| NaOH treated Pt | 15 ± 4° |
| GPTS/TS treated Pt | 62 ± 3° |

The results demonstrated that silane was successfully attached to the sheets by using GPTS/TS, as indicated by the dramatic change in the water contact angle.

### Example 2

Chromium Nickel alloy (Cr/Ni)) sheets were prepared for coatings similar to Example 1 above. In this example, after hydroxylation as described above in Example 1, two sets of Cr/Ni sheets were immersed in silane solutions. The first silane solution contained 1 mL 2-bromo-2-methyl-N-3-[trimethoxysilyl)propyl]-propanamide (BrTMOS), 37.5 mL absolute ethanol, and 1.5 mL deionized water. The second silane solution contained 750 μl BrTMOS, 250 μl TS, 37.5 mL absolute ethanol, and 1.5 mL deionized water.

After formation of the silane layer with either BrTMOS or the combination of BrTMOS and TS, an additional coating including 2-methacryloyloxyethyl phosphorylcholine (MPC) was applied by submerging the treated Cr/Ni sheets into MPC monomer solution with stirring for 4 hours at room temperature. The MPC monomer solution included 500 mg MPC, 28.6 mg of Copper (I) bromide, and 62.5 mg of 2,2-dipyridyl in 300 mL of ethanol.

XPS was conducted as described above in Example 1. The ratio of phosphorus to chromium on the sheets possessing the BrTMOS silane layer was 0.15, while the ratio of phosphorus to chromium on the sheets possessing the BrTMOS and TS as the silane layer was 0.30.

### Example 3

Stents were coated with the BrTMOS silane layer and the combination BrTMOS and TS silane layer following the process described above in Example 2. The stents were braided PIPELINE™ embolization devices, commercially available from Covidien, Mansfield, Mass.

The stents were subjected to a thrombogram test, in which thrombin formation was measured by detecting the fluorescence of a thrombin-sensitive fluorescent additive in a test solution containing a sample of the stent. The thrombogram proceeded as follows. Citrated, aphorized, human platelet-rich plasma (PRP; American Red Cross, Dedham, Mass.) was centrifuged at 2900×g to obtain platelet poor plasma (PPP). Platelet count in PRP was adjusted to 200,000 per microliter by adding PPP. To this diluted PRP, the following reagents were added: a thrombin specific fluorogenic substrate (Z-Gly-Gly-Arg-AMC-HCl, catalog no. 1-1140, Bachem Americas Inc., Torrance, Calif.) to a final concentration of 400 μM and calcium chloride (catalog no. 223506-2.5KG, Sigma Aldrich, St. Louis, Mo.) to a final concentration of 20 mM. A calibration mixture was prepared by adding reference thrombin calibrator solution (catalog no. TS20.00, Stago Diagnostics Inc., Parsippany, N.J.) and the fluorogenic substrate to the PPP, in proportions of 1 part substrate to 11 parts calibrator solution to 98 parts PPP, arriving at a final concentration of the fluorogenic substrate of about 400 μM. Samples of stents prepared according to this Example 3 (“test stents”) and of identical, but bare-metal stents (“bare stents”), were prepared by cutting sections of each stent to a length of 9 mm. The 9 mm sections of test stents and bare stents were placed individually in separate wells of a black, opaque 96-well microplate (Fisher Scientific, Waltham, Mass.). Test solution (330 microliters) was added to each well containing a test stent or bare stent sample, to several wells each containing a 4 mm glass sphere (Fisher Scientific, Cat. No. 11-312B) to serve as a positive control, and to several empty wells to serve as a negative control. Calibration mixture (330 microliters) was added to several empty wells (separate from the negative-control wells) to provide a calibration reference. Fluorescence was measured in a SYNERGY™ HT microplate reader (BioTek, VT) with the following settings: test temperature 25° C.; excitation at 360 nm; emission at 460 nm; no shake, kinetic readings at intervals of 1 minute for each well; and total experimental time of 2 hours.

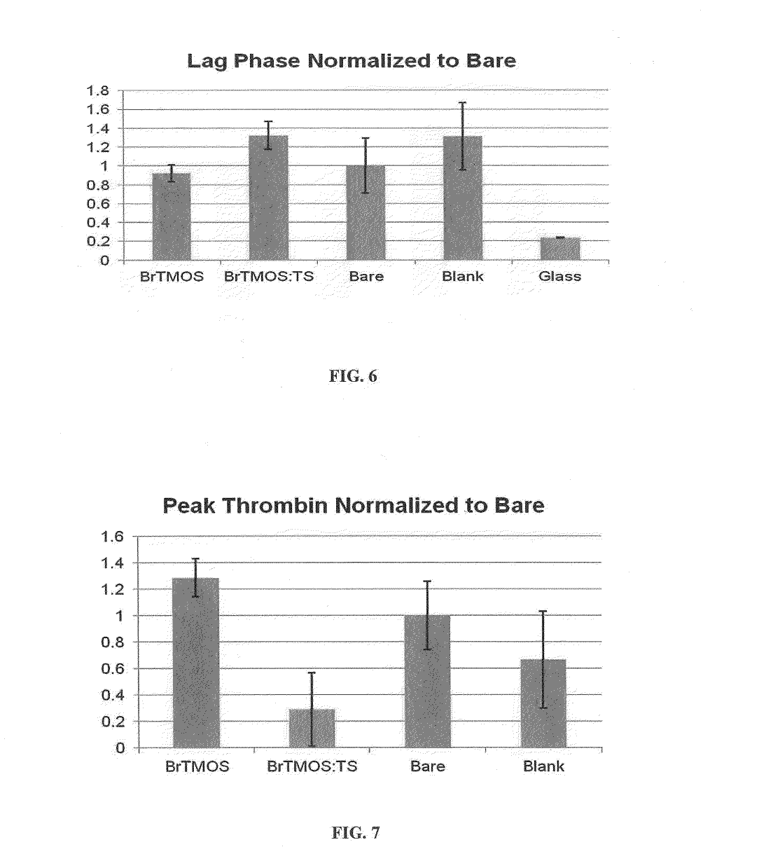

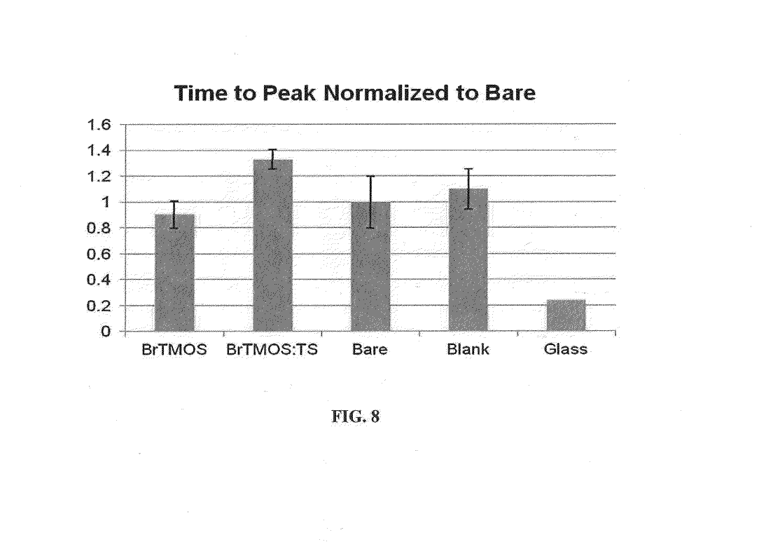

The lag phase, i.e., the time before the onset of thrombin formation, was measured with the results set forth in FIG. 6. The peak thrombin concentration was also measured, with the results set forth in FIG. 7. The rate of thrombin formation was also measured by the time before peak thrombin concentration, with the results set forth in FIG. 8. Indicators that coatings were more efficacious, and less thrombogenic, included an increase in lag time, decrease in peak thrombin concentration, and a decrease in rate of thrombin formation indicated by an increase in time before peak thrombin concentration.

The coated stents were compared to the bare stents, the glass spheres, and the empty polystyrene wells. The data results were normalized to the bare stent. As can be seen from the data in FIGS. 6-8, stents coated with BrTMOS and TS as the silane layer exhibited the best results, with a longer lag time (about 30% longer, FIG. 6), lower magnitude of peak thrombin (about 75% lower, FIG. 7), and a longer time to peak thrombin formation (about 30% longer, FIG. 8).

### Methods of Treatment

The present disclosure also includes methods of treating a vascular condition, such as an aneurysm or intracranial aneurysm, with any of the embodiments of the coated or surface-streated stents disclosed herein. The coated/surface-treated, low-thrombogenicity stent can be deployed across the neck of an aneurysm and its flow-diverting properties employed to reduce blood flow between the aneurysm and the parent vessel, cause the blood inside the aneurysm to thrombose and lead to healing of the aneurysm.

Significantly, the low-thrombogenicity stents disclosed herein can facilitate treatment of a large population of patients for whom flow-diverter therapy has not been previously possible. Such patients are those who have previously suffered from a hemorrhagic aneurysm or who have been diagnosed as being at risk for hemorrhage from an aneurysm in the intracranial arterial system. These patients cannot currently be treated with commercially available flow-diverting stents because those stents are bare metal, braided stents whose implantation requires the patient to take blood-thinning medication (typically aspirin and PLAVIX™ (clopidogrel)) for a long period of time following implantation. The purpose of the blood-thinning medication is to counteract the tendency of the bare-metal stent to cause thrombus (blood clots) to form in the patient's vasculature. However, for a patient who has suffered from, or is at risk of, intracranial hemorrhage, taking the blood-thinning medication can cause, or put the patient at higher risk of, such a hemorrhage. Low-thrombogenicity flow-diverting stents, such as the coated stents disclosed herein, can make flow-diverter therapy possible for patients who cannot tolerate blood-thinning medication because the reduced thrombogenicity can reduce or eliminate the need for blood thinners.

In order to implant any of the coated stents disclosed herein, the stent can be mounted in a delivery system. Suitable delivery systems are disclosed in U.S. patent application Ser. No. 13/692,021, filed Dec. 3, 2012, titled METHODS AND APPARATUS FOR LUMINAL STENTING; and in U.S. Pat. No. 8,273,101, issued Sep. 25, 2012, titled SYSTEM AND METHOD FOR DELIVERING AND DEPLOYING AN OCCLUDING DEVICE WITHIN A VESSEL. The entire contents of both of these documents are incorporated by reference herein and made a part of this specification. In particular, these documents' teachings regarding stent delivery systems and methods may be employed to deliver any of the coated stents disclosed herein in the same manner, to the same bodily location(s), and using the same components as are disclosed in both incorporated documents.

Generally, the delivery system can include an elongate core assembly that supports or contains the stent, and both components can be slidably received in a lumen of a microcatheter or other elongate sheath for delivery to any region to which the distal opening of the microcatheter can be advanced. The core assembly is employed to advance the stent through the microcatheter and out the distal end of the microcatheter so that the stent is allowed to self-expand into place in the blood vessel, across an aneurysm or other treatment location.

A treatment procedure can begin with obtaining percutaneous access to the patient's arterial system, typically via a major blood vessel in a leg or arm. A guidewire can be placed through the percutaneous access point and advanced to the treatment location, which can be in an intracranial artery. The microcatheter is then advanced over the guidewire to the treatment location and situated so that a distal open end of the microcatheter is adjacent to the treatment location. The guidewire can then be withdrawn from the microcatheter and the core assembly, together with the stent mounted thereon or supported thereby, can be advanced through the microcatheter and out the distal end thereof. The stent can then self-expand into apposition with the inner wall of the blood vessel. Where an aneurysm is being treated, the stent is placed across the neck of the aneurysm so that a sidewall of the stent (e.g. a section of the braided tube) separates the interior of the aneurysm from the lumen of the parent artery. Once the stent has been placed, the core assembly and microcatheter are removed from the patient. The stent sidewall can now perform a flow-diverting function on the aneurysm, thrombosing the blood in the aneurysm and leading to healing of the aneurysm.

Because of the low-thrombogenic properties of the coated stents disclosed herein, certain additional aspects of the methods of treatment are possible. For example, the patient can be one who has previously suffered from, or who has been diagnosed as being at risk of, hemorrhage from an aneurysm in arterial anatomy such as the intracranial arterial system. The patient can be prescribed a reduced regimen of blood-thinning medication as compared to the regimen that would be necessary for a patient who received an otherwise similar but uncoated flow-diverting stent. The regimen can be “reduced” in the sense that the patient takes a lower dosage, fewer medications, less powerful medications, follows a lower dosage frequency, and/or takes medication for a shorter period of time following implantation of the stent, or otherwise. Alternatively, the patient may be prescribed no blood thinning medication at all.

The devices and methods discussed herein are not limited to the coating of stents, but may include any number of other implantable devices. Treatment sites may include blood vessels and areas or regions of the body such as organ bodies.

The compositions and methods specifically described herein are non-limiting exemplary embodiments, and that the description, disclosure, and Figures should be construed merely as exemplary of particular embodiments. It is to be understood, therefore, that the present disclosure is not limited to the precise embodiments described, and that various other changes and modifications may be effected without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

What is claimed is:

1. A medical device, comprising:
a substrate;
a silane layer comprising at least one sulfur-functional silane and at least one additional silane, on at least a portion of the substrate;
at least one additional component bound to the silane layer, the at least one additional component selected from the group consisting of monomers, polymers, bioactive agents, and combinations thereof.

2. The medical device of claim 1, wherein the substrate comprises an inert material selected from the group consisting of glass, ceramics, and metals.

3. The medical device of claim 2, wherein the substrate comprises a metal selected from the group consisting of gold, silver, copper, steel, aluminum, cobalt, chromium, platinum, titanium, niobium, tantalum, alloys thereof, and combinations thereof.

4. The medical device of claim 1, wherein the at least one sulfur-functional silane is selected from the group consisting of bis-[triethoxysilylpropyl]tetrasulfide, 3-mercaptopropyltriethoxysilane, 2,2-dimethoxy-1-thia-2-silacyclo-pentane, 11-mercaptoundecyltrimethoxysilane,s-(octanoyl)mercaptopropyl-triethoxysilane, 2-(2-pyridylethyl)thiopropyltri-methoxysilane, 2-(4-pyridylethyl)thiopropyltri-methoxysilane, 3-thiocyanatopropyltriethoxysilane, 2-(3-trimethoxysilylpropylthio)-thiophene, mercaptomethylmethyldiethoxy-silane, 3-mercaptopropylmethyldimethoxy-silane, bis[3-(triethoxysilyl)propyl]-disulfide, bis-[m-(2-triethoxysilylethyl)tolyl]-polysulfide, bis[3-(triethoxysilyl)propyl]thio-urea, bis-triethoxy silyl propyl polysulfide, and combinations thereof.

5. The medical device of claim 1, wherein the at least one additional silane possesses functional groups selected from the group consisting of acrylates, methacrylates, aldehydes, amino, epoxy, esters, anhydride, azide, carboxylate, phosphonate, sulfonate, halogen, hydroxyl, isocyanate, masked isocyanate, phosphine, phosphate, vinyl, olefin, dipodal silanes, UV active components, fluorescent components, chiral components, biomolecular probes, silyl hydrides and combinations thereof.

6. The medical device of claim 1, wherein the at least one sulfur-functional silane is present in amounts from about 0.5% to about 95% by weight of the silane layer.

7. The medical device of claim 1, wherein the at least one additional silane is present in amounts from about 99.5% to about 5% by weight of the silane layer.

8. The medical device of claim 1, wherein the at least one additional component bound to the silane layer is selected from the group consisting of betaines, phosphorylcholines, [2-(Methacryloyloxy)ethyl]dimethyl-(3-sulfopropyl)ammonium hydroxide, 3-(Methacryloylamino)propyl]dimethyl(3-sulfopropyl)ammonium hydroxide inner salt, and combinations thereof.

9. The medical device of claim 1, wherein the at least one additional component bound to the silane layer comprises a phosphorylcholine selected from the group consisting of 2-methacryloyloxyethyl phosphorylcholine, 2-acryloyloxyethyl phosphorylcholine, 2-acryloyloxyethyl phosphorylcholine, 2-(meth)acryloyloxyethyl-2′-(trimethylammonio)ethyl phosphate, 3-(meth)acryloyloxypropyl-2′-(trimethylammonio)ethyl phosphate, 4-(meth)acryloyloxybutyl-2′-(trimethylammonio)ethyl phosphate, 5-(meth)acryloyloxypentyl-2′-(trimethylammonio)ethyl phosphate, 6-(meth)acryloyloxyhexyl-2′-(trimethylammonio)ethyl phosphate, 2-(meth)acryloyloxyethyl-2′-(triethylammonio)ethyl phosphate, 2-(meth)acryloyloxyethyl-2′-(tripropylammonio)ethyl phosphate, 2-(meth)acryloyloxyethyl-2′-(tributylammonio)ethyl phosphate, 2-(meth)acryloyloxypropyl-2′-(trimethylammonio)ethyl phosphate, 2-(meth)acryloyloxybutyl-2′-(trimethylammonio)ethyl phosphate, 2-(meth)acryloyloxypentyl-2′-(trimethylammonio)ethyl phosphate, 2-(meth)acryloyloxyhexyl-2′-(trimethylammonio)ethyl phosphate, 2-(meth)acryloyloxyethyl-3′-(trimethylammonio)propyl phosphate, 3-(meth)acryloyloxypropyl-3′-(trimethylammonio)propyl phosphate, 4-(meth)acryloyloxybutyl-3′-(trimethylammonio)propyl phosphate, 5-(meth)acryloyloxypentyl-3′-(trimethylammonio)propyl phosphate, 6-(meth)acryloyloxyhexyl-3′-(trimethylammonio)propyl phosphate, 2-(meth)acryloyloxyethyl-4′-(trimethylammonio)butyl phosphate, 3-(meth)acryloyloxypropyl-4′-(trimethylammonio)butyl phosphate, 4-(meth)acryloyloxybutyl-4′-(trimethylammonio)butyl phosphate, 5-(meth)acryloyloxypentyl-4′-(trimethylammonio)butyl phosphate, 6-(meth)acryloyloxyhexyl-4′-(trimethylammonio)butylphosphate, and combinations thereof.

10. The medical device of claim 1, wherein the at least one additional component is chemically bonded to the silane layer.

11. The medical device of claim 1, wherein the at least one additional component is covalently bonded to the silane layer.

12. The medical device of claim 1, wherein the at least one additional component comprises a bioactive agent selected from the group consisting of antimicrobials, analgesics, anesthetics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, steroids, lipids, lipopolysaccharides, polysaccharides, enzymes, and combinations thereof.

13. The medical device of claim 1, wherein the medical device is selected from the group consisting of stents, filters, stent coatings, grafts, catheters, stent/grafts, clips and other fasteners, staples, sutures, pins, screws, prosthetic devices, drug delivery devices, anastomosis rings, surgical blades, contact lenses, intraocular lenses, surgical meshes, knotless wound closures, sealants, adhesives, intraocular lenses, anti-adhesion devices, anchors, tunnels, bone fillers, synthetic tendons, synthetic ligaments, tissue scaffolds, stapling devices, buttresses, lapbands, orthopedic hardware, pacers, pacemakers, and implants.

14. The medical device of claim 1, wherein the medical device comprises a stent.

15. A method for forming a silane layer on a surface of a medical device comprising:
contacting the surface of the medical device with at least one sulfur-functional silane and at least one additional silane to form the silane layer, and
contacting the silane layer with at least one additional component selected from the group consisting of monomers, polymers, bioactive agents, and combinations thereof.

16. The method of claim 15, wherein the at least one sulfur-functional silane is selected from the group consisting of bis-[triethoxysilylpropyl]tetrasulfide, 3-mercaptopropyltriethoxysilane, 2,2-dimethoxy-1-thia-2-silacyclo-pentane, 11-mercaptoundecyltrimethoxysilane, s-(octanoyl)mercaptopropyl-triethoxysilane, 2-(2-pyridylethyl)thiopropyltri-methoxysilane, 2-(4-pyridylethyl)thiopropyltri-methoxysilane, 3-thiocyanatopropyltriethoxysilane, 2-(3-trimethoxysilylpropylthio)-thiophene, mercaptomethylmethyldiethoxy-silane, 3-mercaptopropylmethyldimethoxy-silane, bis[3-(triethoxysilyl)propyl]-disulfide, bis-[m-(2-triethoxysilylethyl)tolyl]-polysulfide, bis[3-(triethoxysilyl)propyl]thio-urea, bis-triethoxy silyl propyl polysulfide, and combinations thereof.

17. The method of claim 15, wherein the at least one additional silane possesses functional groups selected from the group consisting of acrylates, methacrylates, aldehydes, amino, epoxy, esters, anhydride, azide, carboxylate, phosphonate, sulfonate, halogen, hydroxyl, isocyanate, masked isocyanate, phosphine, phosphate, vinyl, olefin, dipodal silanes, UV active components, fluorescent components, chiral components, biomolecular probes, silyl hydrides, and combinations thereof.

18. The method of claim 15, further comprising hydroxylating the surface of the medical device prior to contacting the surface with the at least one sulfur-functional silane and the at least one additional silane.

19. The method of claim 18, wherein the surface of the medical device is hydroxylated by subjecting the surface to a treatment selected from the group consisting of sodium hydroxide, nitric acid, sulfuric acid, hydrochloric acid, ammonium hydroxide, hydrogen peroxide, tert-butyllyn hydroperoxide, potassium dichromate, perchloric acid, oxygen plasma, water plasma, corona discharge, ozone, UV, and combinations thereof.

20. The method of claim 15, wherein the at least one sulfur-functional silane is present in amounts from about 0.5% to about 95% by weight of the silane layer.

21. The method of claim 15, wherein the at least one additional silane is present in amounts from about 99.5% to about 5% by weight of the silane layer.

22. The method of claim 15, wherein the at least one sulfur-functional silane and the at least one additional silane are sequentially applied.

23. The method of claim 15, wherein the at least one sulfur-functional silane and the at least one additional silane are applied in a single mixture.

24. The method of claim 15, wherein the at least one additional component bound to the silane layer is selected from the group consisting of betaines, phosphorylcholines, [2-(Methacryloyloxy)ethyl]dimethyl-(3-sulfopropyl)ammonium hydroxide, 3-(Methacryloylamino)propyl]dimethyl(3-sulfopropyl)ammonium hydroxide inner salt, and combinations thereof.

25. The method of claim 15, wherein the at least one additional component bound to the silane layer comprises a phosphorylcholine selected from the group consisting of 2-methacryloyloxyethyl phosphorylcholine, 2-acryloyloxyethyl phosphorylcholine, 2-acryloyloxyethyl phosphorylcholine, 2-(meth)acryloyloxyethyl-2′-(trimethylammonio)ethyl phosphate, 3-(meth)acryloyloxypropyl-2′-(trimethylammonio)ethyl phosphate, 4-(meth)acryloyloxybutyl-2′-(trimethylammonio)ethyl phosphate, 5-(meth)acryloyloxypentyl-2′-(trimethylammonio)ethyl phosphate, 6-(meth)acryloyloxyhexyl-2′-(trimethylammonio)ethyl phosphate, 2-(meth)acryloyloxyethyl-2′-(triethylammonio)ethyl phosphate, 2-(meth)acryloyloxyethyl-2′-(tripropylammonio)ethyl phosphate, 2-(meth)acryloyloxyethyl-2′-(tributylammonio)ethyl phosphate, 2-(meth)acryloyloxypropyl-2′-(trimethylammonio)ethyl phosphate, 2-(meth)acryloyloxybutyl-2′-(trimethylammonio)ethyl phosphate, 2-(meth)acryloyloxypentyl-2′-(trimethylammonio)ethyl phosphate, 2-(meth)acryloyloxyhexyl-2′-(trimethylammonio)ethyl phosphate, 2-(meth)acryloyloxyethyl-3′-(trimethylammonio)propyl phosphate, 3-(meth)acryloyloxypropyl-3′-(trimethylammonio)propyl phosphate, 4-(meth)acryloyloxybutyl-3′-(trimethylammonio)propyl phosphate, 5-(meth)acryloyloxypentyl-3′-(trimethylammonio)propyl phosphate, 6-(meth)acryloyloxyhexyl-3′-(trimethylammonio)propyl phosphate, 2-(meth)acryloyloxyethyl-4′-(trimethylammonio)butyl phosphate, 3-(meth)acryloyloxypropyl-4′-(trimethylammonio)butyl phosphate, 4-(meth)acryloyloxybutyl-4′-(trimethylammonio)butyl phosphate, 5-(meth)acryloyloxypentyl-4′-(trimethylammonio)butyl phosphate, 6-(meth)acryloyloxyhexyl-4′-(trimethylammonio)butylphosphate, and combinations thereof.

26. The method of claim 15, wherein the at least one additional component is chemically bonded to the silane layer.

27. The method of claim 15, wherein the at least one additional component is covalently bonded to the silane layer.

28. The method of claim 15, wherein the at least one additional component comprises a bioactive agent selected from the group consisting of antimicrobials, analgesics, anesthetics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, steroids, lipids, lipopolysaccharides, polysaccharides, enzymes, and combinations thereof.

29. The method of claim 15, wherein the medical device is selected from the group consisting of stents, filters, stent coatings, grafts, catheters, stent/grafts, clips and other fasteners, staples, sutures, pins, screws, prosthetic devices, drug delivery devices, anastomosis rings, surgical blades, contact lenses, intraocular lenses, surgical meshes, knotless wound closures, sealants, adhesives, intraocular lenses, anti-adhesion devices, anchors, tunnels, bone fillers, synthetic tendons, synthetic ligaments, tissue scaffolds, stapling devices, buttresses, lapbands, orthopedic hardware, pacers, pacemakers, and implants.

30. The method of claim 15, wherein the medical device comprises a stent.
